(19) United States
(12) Patent Application Publication
(10) Pub. No.: US 2010/0305407 A1
(43) Pub. Date: Dec. 2, 2010

(54) Malleable Port Retractor

(76) Inventor: Daniel K. Farley, Traverse City, MI (US)

Correspondence Address: MCANDREWS HELD & MALLOY, LTD, 500 WEST MADISON STREET, SUITE 3400, CHICAGO, IL, 60661, US

(21) Appl. No.: 12/792,313

(22) Filed: Jun. 2, 2010

Related U.S. Application Data
(60) Provisional application No. 61/183,164, filed on Jun. 2, 2009.

(51) Int. Cl. A61B 1/32

(52) U.S. Cl. 600/206

(57) ABSTRACT

A port retractor includes malleable leaves that may be bent outwardly to an open position from a closed position. The port retractor includes a body having a cross-section defining a substantially closed area, with the leaves extending therefrom. The leaves extend from the body at an angle when bent to the open position. Further, the port retractor may include opposed pairs of leaves.

### CROSS-REFERENCE TO RELATED APPLICATION

The present application makes reference to, claims priority to, and claims benefit of U.S. Provisional Application No. 61/183,164, entitled “Malleable Port Retractor,” Attorney Docket No. 20568US01, filed Jun. 2, 2009, the complete subject matter of which is hereby incorporated herein by reference, in its entirety.

### STATEMENT REGARDING FEDERALLY SPONSORED RESEARCH OR DEVELOPMENT

None.

### BACKGROUND OF THE INVENTION

The present invention relates to surgical apparatus for retracting anatomy to provide exposure of an operative site, and more particularly relates to a retraction apparatus having a port with malleable features.

In surgical operations, retraction devices are used to properly access internal organs and bone structures. Retraction devices are generally designed to hold back the anatomy in the immediate area of the operative site to enable a surgeon to have both an optimal view of the site and a sufficiently open area within which to work. During a surgical procedure, a surgeon will typically make an incision in a patient to access the sites of interest, such as an internal organ or organs, and/or bone structures, depending on the procedure. A retraction device may then be used to maintain clear access to the site of interest.

For example, during a spinal fixation procedure, screws are inserted into a patient's vertebrae, typically at the pedicles. A rod may then be secured to the screws, thereby helping to fix a particular potion of the spinal column in place. To perform such a procedure, access to the spinal column is needed. A retractor may be used to hold back tissue and allow a surgeon access to place one or more screws and/or connecting rods, and/or perform other procedures on the spinal column. Ideally, such access would include room to manipulate the various surgical implements required, as well as good lighting and visibility. Further, it may be desirable for such access to allow for adjustability to accommodate different patients or procedures. Moreover, it may further be desirable to have the ability to maneuver around a placed rod and/or other obstruction at the access site.

It is therefore one object of the present invention to provide a surgical retractor that provides improved access, and/or visibility, and/or adjustability, and/or maneuverability around a surgical site of interest.

### BRIEF SUMMARY OF THE INVENTION

These and other objects of the invention are achieved in a port retractor. In one embodiment of the present invention, a port retractor includes malleable, or bendable, leaves extending from a body. One or more of the malleable leaves may be bent outward to an open position from a closed position, such that one or more of the malleable leaves extend outwardly from the body at an angle when bent to the open position. In certain embodiments, in the closed position, the leaves may be substantially aligned with the body. In certain embodiments, in the closed position, the leaves may extend inwardly from the body, such that a cross-sectional area defined by the leaves at a distal end is smaller than a cross-sectional area defined by the leaves at a more proximal location. Further, the port retractor may include opposed pairs of leaves.

In certain embodiments, the port retractor includes a sheath that is attachable to and removable from at least a portion of the leaves. The sheath surrounds at least a portion of the distal portion of the leaves when attached to the port retractor. The sheath is expandable with the port retractor when the leaves are moved from the closed position to the open position and collapsible with the port retractor when the leaves are moved from the open position to the closed position.

In certain embodiments, the retractor system includes a leaf bender tool. The port retractor may include openings in the leaves adapted for insertion of the leaf bender tool for bending the leaves.

In one embodiment of the present invention, a method of providing access to a surgical incision with a port retractor is provided. The method includes positioning the port retractor, in a closed position, inside the patient. The method also includes securing the port retractor in place and adjusting the port retractor to an open position by bending leaves outwardly. In certain embodiments, the method may include surrounding at least a portion of a distal portion of the port retractor with an expandable sheath before positioning the port retractor inside the patient. Further, the method may comprise utilizing a leaf bender to bend at least one leaf of the port retractor after the port retractor is positioned inside the patient.

### BRIEF DESCRIPTION OF THE SEVERAL VIEWS OF THE DRAWINGS

FIG. 1 provides a side view of a port retractor formed in accordance with an embodiment of the present invention in a closed position.

FIG. 2 provides a side view of the port refractor of FIG. 1 in an open position.

FIG. 3 provides a top view of the port retractor of FIG. 1 in a closed position.

FIG. 4 provides a top view of a port retractor formed in accordance with an embodiment of the present invention in a closed position.

FIG. 5 provides a side view of the port retractor of FIG. 4.

FIG. 6 provides a perspective view of a sheath foamed in accordance with an embodiment of the present invention.

FIG. 7 provides a side view of a leaf bender formed in accordance with an embodiment of the present invention.

FIG. 8 provides a side view of a leaf bender formed in accordance with an embodiment of the present invention.

FIG. 9 provides a side view of a port refractor formed in accordance with an embodiment of the present invention in a closed position.

FIG. 10 provides a side view of the port refractor of FIG. 9 in an open position.

FIG. 11 provides a top view of the port retractor FIG. 9 in a closed position.

FIG. 12 provides a top view of the port refractor FIG. 9 in an open position.

FIG. 13 provides a side view of a distal tip of a leaf of a port refractor formed in accordance with an embodiment of the present invention.

FIG. 14 provides a side view of a distal tip of a leaf of a port retractor foamed in accordance with an embodiment of the present invention.

FIG. 15 provides a side view of a distal tip of a leaf of a port retractor formed in accordance with an embodiment of the present invention.

### DETAILED DESCRIPTION

Aspects of the present invention may be seen in an embodiment of a surgical port retractor system. The surgical port retractor system may include a port retractor 20, a sheath 30, and a bending tool 40.

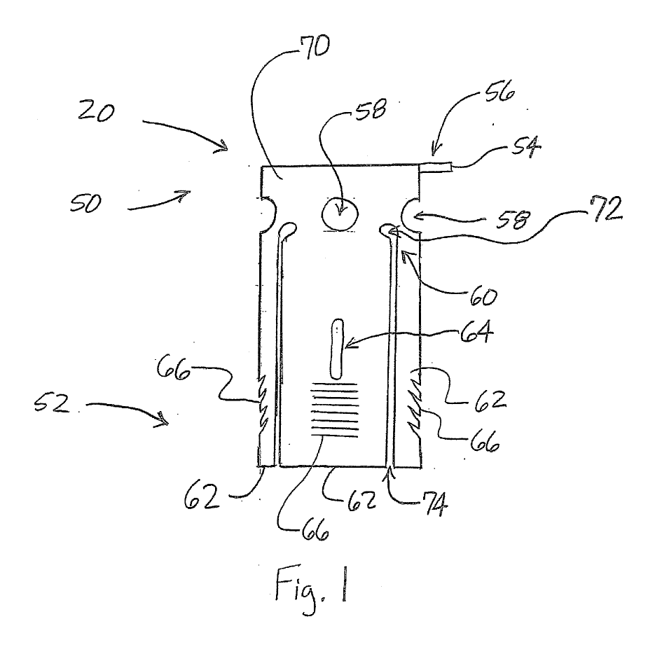

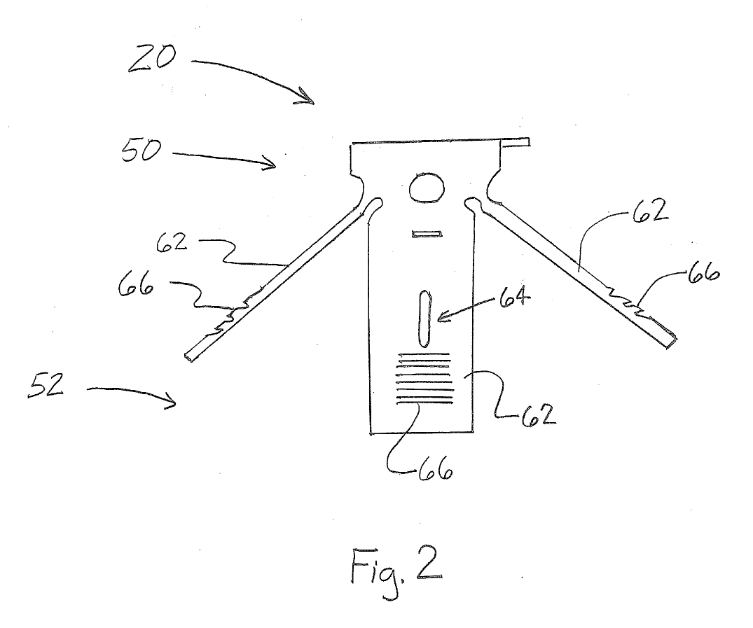

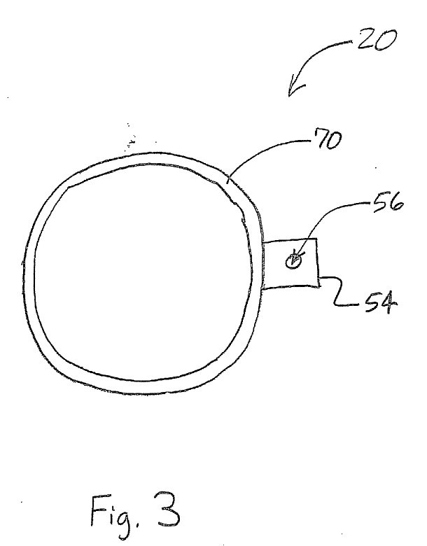

FIG. 1 illustrates a side view of a port retractor 20 in a first, or closed, position, and FIG. 2 illustrates the port retractor 20 in a second, or open, position. FIG. 3 illustrates a top view of the port retractor 20 in a closed position. The port retractor 20 includes a proximal end 50 and a distal end 52. The proximal end 50 is oriented more closely to a practitioner performing a procedure with the port retractor 20 in place in a patient, and the distal end 52 is oriented more deeply inside a patient when the port retractor 20 is in place during a procedure.

The port retractor 20 may be made of, for example, titanium. The port retractor 20 is sized and configured so that portions of it are malleable enough to be bent to suit during a procedure, but rigid enough to maintain a desired shape and hold back tissue during a procedure once bent to suit.

The port retractor 20 includes a body 70, a mounting tab 54, a mounting opening 56, relief openings 58, leaf openings 60, leaves 62, bending openings 64, and sheath retainers 66. In the illustrated embodiment, the mounting tab 54 extends from a surface proximate to the proximal end 50. The mounting tab 54 includes a mounting opening 56. The mounting opening 56 is sized and adapted to allow mounting of the port retractor 20. For example, the mounting opening 56 may accept a rod and/or a universal clamp (not shown) that is in turn secured to a frame that is fixed to, for example, a hospital bed. In alternate embodiments, the port retractor 20 may include different mounting features. As one example, a port retractor may include a mounting post extending from a surface of the port retractor that is accepted by a clamp to secure the port retractor in place during a procedure. Further, the mounting tab 54 may provide a surface with which a practitioner may handle and manipulate the port retractor 20 while positioning the port retractor 20 in place for a procedure, and/or to aid in removing the port retractor 20 after completion of the procedure.

In the embodiment illustrated in FIGS. 1-3, each of the relief openings 58 is generally circularly shaped and located proximate to and between the ends of adjacent leaf openings 60. Thus, each of the relief openings 58 is associated with a corresponding leaf 62, and generally positioned proximate to a base of a leaf 62. The relief openings 58 are sized and positioned to allow easier bending of the leaves 62 by a practitioner during a procedure. By removing material proximate to the base at which a leaf is to be bent, easier bending of the leaf is accomplished while allowing portions of the port retractor that are not desired to be bent to be more rigid. Alternatively, the relief openings 58 may be of different sizes and/or shapes than illustrated in FIG. 1, and/or the sizes and/or shapes of the relief openings 58 may vary in the same port retractor. For example, if it is anticipated that certain of the leaves in a port retractor are anticipated to be bent more frequently and/or to a larger angle, those leaves may have associated with them larger and/or differently shaped relief openings than those associated with leaves that are expected to be bent less frequently or to a lesser extent. As an additional example, if certain leaves are expected to be bent to a smaller degree or not at all, those leaves may have smaller relief openings associated with them, or no relief opening at all. As a further alternative, in alternate embodiments the relief openings may not necessarily be associated with specific leaves. For example, the relief openings may include a series of slots around the exterior of the port retractor. As a further alternative, a port retractor may include a relieved area that does not provide an opening entirely through the wall of the port retractor. For example, material may be removed to form a ring of reduced thickness around the exterior of the port retractor proximate to the bases of the leaves.

In the illustrated embodiment, each leaf opening 60 includes a leaf base opening 72 and a leaf gap 74. As illustrated in FIG. 1, each leaf base opening 72 is generally circularly shaped, and positioned proximate to a side of a leaf 62 proximate to the base of the leaf 62. In the illustrated embodiment, each leaf gap 74 extends distally from a leaf base opening 72 through the distal end 52 of the port retractor 20, thereby separating each leaf 62 from adjacent leaves 62. Thus, each of the leaves 62 may be bent independently of the other leaves 62. In alternate embodiments, different shapes and sizes of leaf base openings 72 and/or leaf gaps 74 may be used, and/or the sizes and/or shapes of the leaf base openings 72 and/or leaf gaps 74 may vary in the same port retractor, similar to the above discussion with respect to the relief openings.

The leaves 62 extend from the body and provide a surface with which to hold back portions of the anatomy from the site of interest. By bending the leaves 62 after insertion, access to the site of interest may be increased, while allowing a relatively smaller cross-section for insertion and placement of the port retractor 20 in a closed position (with the leaves in an unbent position). Thus, the port retractor 20 and various features thereof (such as relief openings and/or leaf openings) should be sized and shaped to provide sufficient rigidity so that the leaves 62 can hold back the desired portions of anatomy, while still allowing sufficient malleability so that the leaves 62 may be bent as desired during a procedure to suit a particular patient and/or procedure. FIG. 2 illustrates a side view of the port retractor 20 illustrated in FIG. 1 with two of its leaves 62 bent to provide improved access to a site of interest during a procedure. In the illustrated embodiment, the leaves 62 are similarly sized and shaped. In alternate embodiments, port retractors may include variously sized and shaped leaves. For example, some leaves on a port retractor may have different widths than others.

In the embodiment illustrated in FIGS. 1-3, each retractor leaf 62 includes a leaf bending opening 64 and a sheath retainer 66. In the embodiment illustrated in FIGS. 1-3, the sheath retainer 66 includes a series of teeth cut into the surface of a leaf 62 proximate to the distal end 52 of the port retractor 20. The sheath retainer 66 acts to prevent the sheath 30 (discussed below) from sliding up the leaf 62, for example, when the leaf 62 is bent outwardly. The leaf bending opening 64 is sized and adapted to accept a feature of a bending tool (discussed below). In the illustrated embodiment the leaf bending opening 64 is an elongated oval shape. The leaf bending opening 64 is sized to accept a feature that is longer along its length then the diameter of the relief openings 58 or the leaf base openings 72, and wider across its width than the leaf gaps 74. This helps prevent the inadvertent placement of the feature in a relief opening 58, leaf base opening 72, or leaf gap 74.

FIG. 3 illustrates a top view of the port retractor 20 illustrated in FIG. 1 in accordance with an embodiment of the present invention. As seen from above, the port retractor 20 includes a body 70 that is generally circularly shaped, with each quadrant of the circle generally corresponding to a leaf 62. Thus, the port retractor 20 illustrated in FIGS. 1-3 includes four leaves, which may also be seen as two opposed pairs of leaves. In the embodiment illustrated in FIG. 3, when in the closed position, the leaves 62 are substantially aligned with the perimeter defined by the body 70. Also, the cross-section of the port retractor 20 may be seen as defining a substantially closed area when in the closed position. For example, in the illustrated embodiment of FIGS. 1-3, at the proximal end 50 of the port retractor 20, the body 70 has a cross-section defining a closed circle, or put another way, the body defines a fixed perimeter having a closed geometric shape, in this case a circle. At the distal end 52, the circle is only interrupted by the relatively thin leaf gaps 74 when in the closed position. Such a use of multiple opposed pairs and/or a cross-section defining a substantially closed area in the closed position allows for improved retractor surface area and positioning. Further, the use of multiple leaves allows for greater adaptability and variety of shapes in the open position. For example, by extending only certain of the leaves, and/or by bending different leaves different amounts, the shape of the retractor in the open position may be quite different from its shape in the closed position. In alternate embodiments, cross-sections that define differently shaped areas such as, for example, substantially oval or polygonal shapes may be used. The polygonal shapes may include, for example, quadrilateral or hexagonal cross-sectional shapes. The polygonal shapes may be equilateral (such as a square) or not equilateral. Further, alternate embodiments of port retractors may include different numbers and/or configurations of leaves. For example, a rectangularly shaped port may include four leaves, one for each segment of the rectangle. As another example, a rectangularly shaped port may include eight leaves, two for each segment of the rectangle.

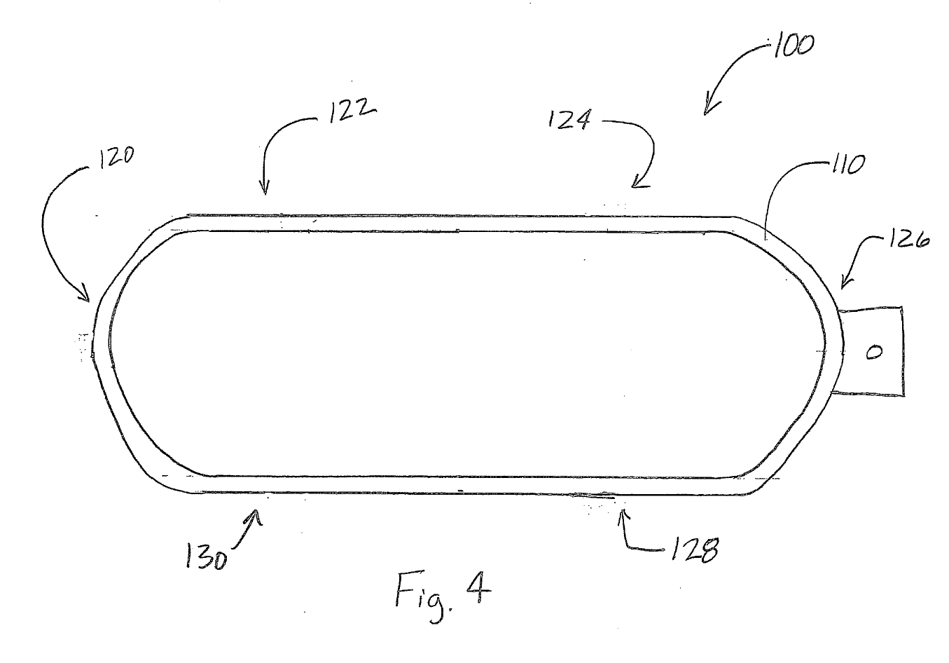

FIG. 4 illustrates a top view of a port retractor 100 formed in accordance with an embodiment of the present invention, and FIG. 5 illustrates a side view of the port retractor 100. The port retractor 100 includes a body 110, a first leaf 120, a second leaf 122, a third leaf 124, a fourth leaf 126, a fifth leaf 128, and a sixth leaf 130. The cross-section of the body 110 defines a substantially oval area. The first leaf 120 is located directly across the length of the oval from the fourth leaf 126. Thus, the first leaf 120 and fourth leaf 124 form an opposed pair. The second leaf 122 and third leaf 124 are directly across the width of the oval from the sixth leaf 130 and fifth leaf 128, respectively, thereby forming two additional opposed pairs. In alternate embodiments, retractor ports may include leaves that do not form opposed pairs, or may include some leaves that form opposed pairs and some leaves that do not.

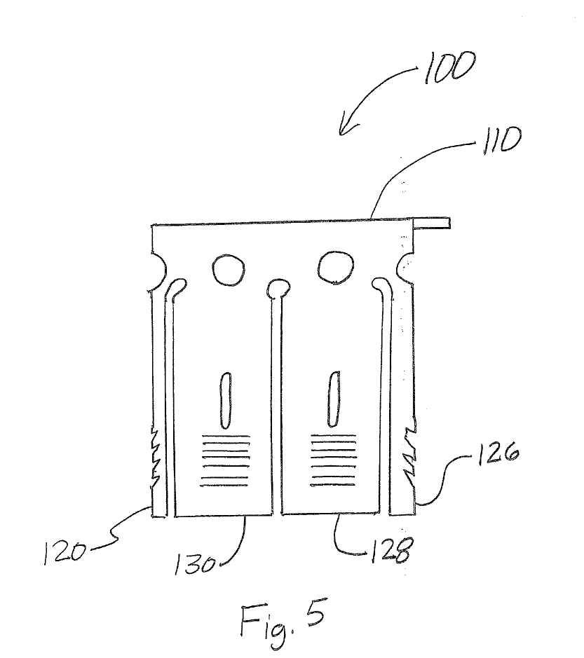

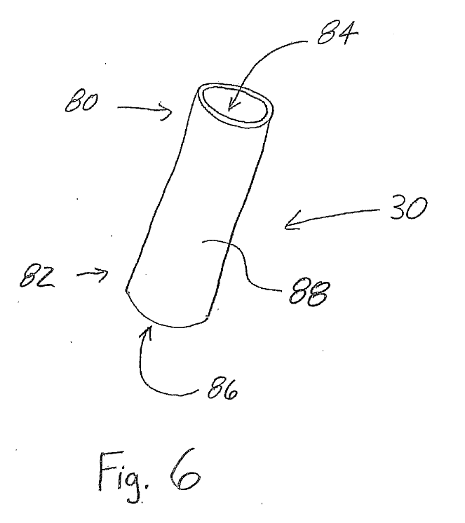

FIG. 6 illustrates a perspective view of a sheath 30 formed in accordance with an embodiment of the present invention. The sheath 30 is used to help prevent or minimize the entry of tissue around the edges of the leaves 62 (or, between gaps between adjacent leaves) of the port retractor 20 into a site of interest, thereby helping provide better access to the site. The sheath 30 illustrated in FIG. 6 includes a proximal end 80, a distal end 82, a first opening 84, a second opening 86, and a wall 88. Regarding the proximal end 80 and a distal end 82, “proximal” and “distal” are generally used as described above. The first opening 84 is located proximate to the proximal end 80, and the second opening 86 is located proximate to the distal end 82. The sheath 30 may made out of an elastic, or stretchable material, with the shape and dimensions of the sheath 30 and the thickness of the wall 88 sized to fit snugly over a port retractor when the port retractor is in the closed position, and to expand with the leaves of the port retractor when the leaves are bent and the port retractor is moved to the open position, while still providing sufficient resiliency to resist the incursion of tissue around the sides of the leaves 62. Thus, the sheath 30 acts to provide a barrier around and between the leaves of the port retractor to help prevent tissue from interfering with access to the site of interest. The sheath 30 may be made, for example, from a latex free surgical grade elastic material. Further, the sheath and leaves may have cooperating surfaces (such as the sheath retainers 66) and/or finishes to help maintain the position of the sheath after it is placed on the port retractor.

FIG. 7 illustrates a side view of a bending tool 200 formed in accordance with an embodiment of the present invention. The bending tool 200 includes a handle 210 and a bending feature 220, and is sized and adapted to provide leverage and accessibility for bending leaves of a port retractor. The bending feature 220 is sized and adapted to be accepted by a leaf bending opening of a port retractor to help align and maintain the bending tool 200 in place with the leaf being bent. For example, the bending feature 220 of a bending tool 200 for use with the port retractor 20 illustrated in FIGS. 1-3 defines an oval slightly smaller than the oval shaped leaf bending opening 64, so that the bending tool 200 may removably engage the leaf 62. The shape of the bending feature 220 for such a bending tool 200 may further be longer along its length then the diameter of the relief openings 58 or the leaf base openings 72, and wider than the leaf gaps 74, to help prevent the inadvertent placement of the bending feature 220 in a relief opening 58, leaf base opening 72, or leaf gap 74.

To bend a leaf 62, the bending tool 200 is inserted into the interior of the port retractor 20, and aligned so that the bending feature 220 is placed in a leaf bending opening 64. The bending tool 200 is then pressed against the inside of the leaf 62, causing the leaf 62 to bend outwardly. The bending feature 220 may also include a hook 222. The hook 222 provides a surface that presses against the exterior of the leaf 62 to help in bending the leaf 62 back toward its original position for removal of the port retractor 20. For example, the bending feature 220 of the bending tool 200 may be inserted into the bending opening of a leaf, and then urged distally so that the open area underneath the hook 222 accepts a portion of a wall of the leaf located distal of the bending opening, thereby providing engagement with both the inner and outer surfaces of the wall so that the bending tool 200 may be used to urge the leaf inwardly as well as outwardly, as desired. In alternate embodiments, different combinations of bending features on the bending tool and mounting features on the port retractor may be used. For example, a post may be located proximate to a distal end of the bending tool, with the post being accepted by a ring on the interior of a leaf. As another example, the interior of the leaf may include a protrusion that is accepted by an opening of a leaf bender.

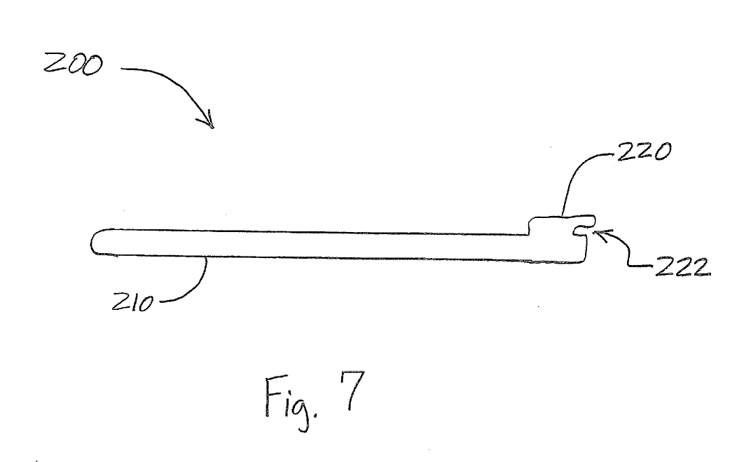

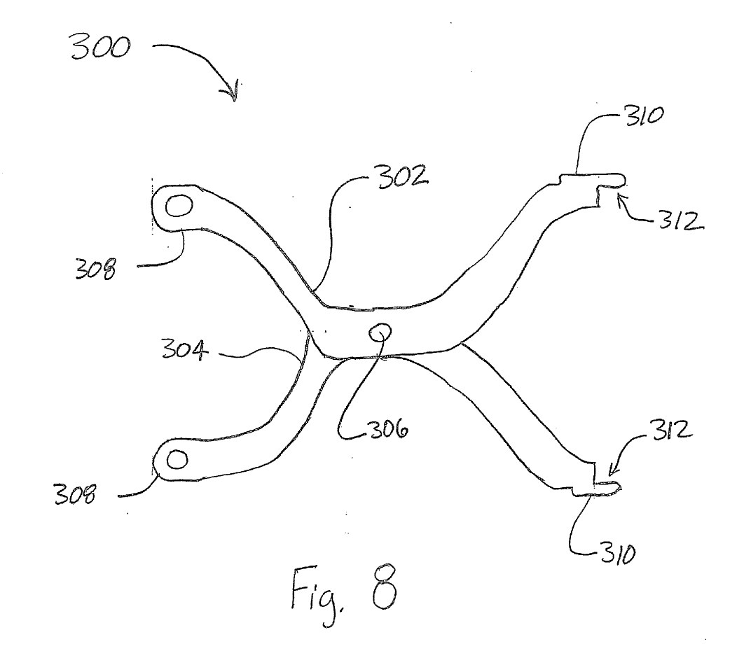

FIG. 8 illustrates a side view of a bending tool 300 formed in accordance with an embodiment of the present invention. The bending tool 300 illustrated in FIG. 8 may be especially useful in bending opposed pairs of leaves. The bending tool 300 includes a first arm 302 and a second aim 304 joined at a pivot 306. The first arm 302 and second arm 304 each include a handle 308 and a bending feature 310. In the illustrated embodiment, the first arm 302 and second arm 304 are generally similar. In alternate embodiments, the first and second arms may be differently shaped and/or include differently sized and/or shaped bending features and/or handles. The handle 308 is sized and adapted to provide a convenient location for a practitioner to grip, and to provide leverage and accessibility for bending leaves of a port retractor. The bending feature 310 is sized and adapted to be accepted by a leaf bending opening of a port retractor. For example, the bending feature 310 may be similarly shaped as described above, and include hooks 312. The bending tool 300 also includes surfaces 314 that contact the interior of leaves to help urge the leaves outwardly when bending a retractor to an open position.

In the illustrated embodiment, the first arm 302 and second arm 304 are configured so that when the handles 308 of the first arm 302 and second arm 304 are brought together, the first arm 302 and second arm 304 move about the pivot 306 such that the bending features 310 of the first arm 302 and second arm 304 are urged apart. To bend opposed leaves 62 of the port retractor 20, the bending tool 300 is inserted into the interior of the port retractor 20, and aligned so that the bending features 310 are each placed in a leaf bending opening 64. The handles 308 of the bending tool 300 are then brought together, urging the bending features 310 away from each other, thereby exerting a force on the inside of two leaves 62, causing them to bend outwardly. To bend leaves back toward the original position for removal of the port retractor 20, the bending features 310 are each placed in a leaf bending opening 64 as before. The handles 308 are then spread apart thereby urging the bending features 310 toward each other, and the hooks 312 provide surfaces that press against the exterior of two leaves 62 to help in bending the leaves 62 back toward their original position for removal of the port retractor 20. In alternate embodiments, the leaf bending tool may be configured more similarly to conventional scissors or pliers, with the bending features being urged together when the handles are brought together. Differently shaped bending tools, or combinations of bending tools, may be included as part of a set to provide for convenient bending of differently sized, shaped, or configured leaves of port retractors, or to provide improved access for a variety of patients and/or procedures.

To use the port retractor 20, a sheath 30, if desired, may first placed over a distal portion of the port retractor 20 in its closed position. After an incision is made, a dilator may be used to enlarge the opening to the site of interest. With the incision at a desired size, the port retractor 20 may then be inserted, distal end first, into the incision and toward the site of interest, and the port retractor 20 may be secured to a frame. Next, the malleable leaves are opened as desired to provide the desired access to the site of interest. For a port retractor having opposed leaves, a device such as bending tool 300 may be used to bend two opposed leaves at once. Alternatively or additionally, a device such as bending tool 200 may be used to bend individual leaves one at a time, and/or to fine tune the positioning of opposed leaves previously bent by a device such as bending tool 300. Once the intended procedure has been performed and access to the site is no longer required, the leaves may be bent back to a reduced cross-section, such as, for example, being substantially returned to the original, closed position, and the port retractor released from the frame and removed from the patient.

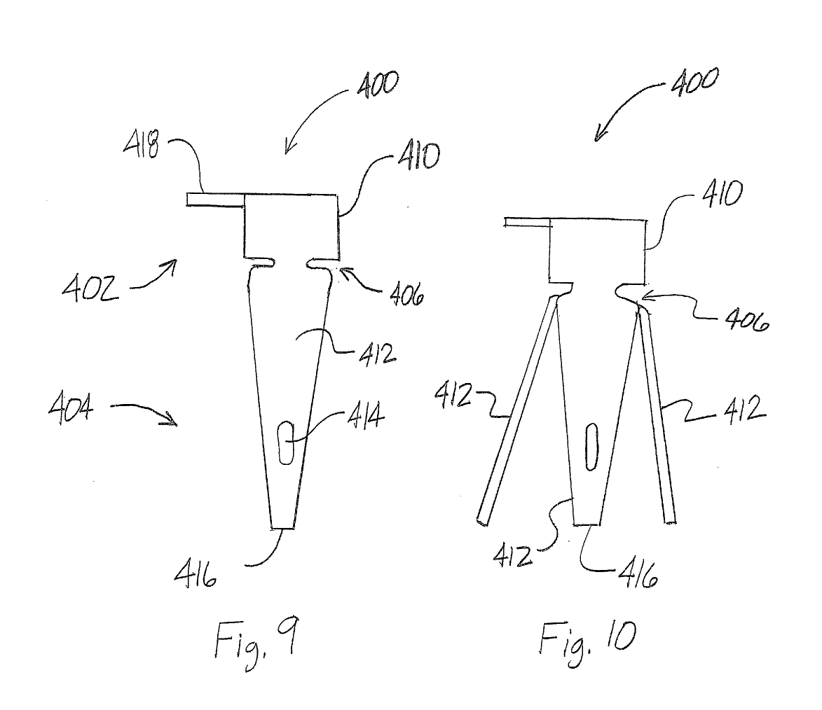

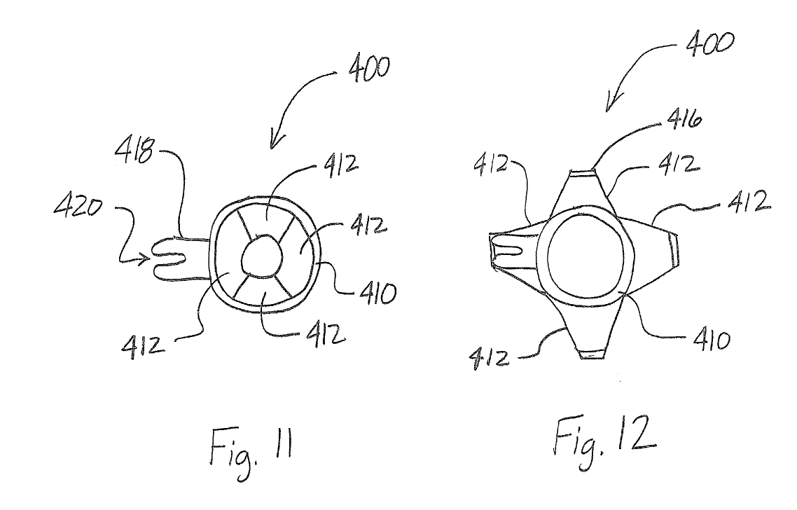

FIG. 9 provides a side view of a port retractor 400 formed in accordance with another embodiment of the present invention in a closed position. FIG. 10 illustrates a side view of the port retractor 400 in an open position, and FIGS. 11 and 12 provide top views of the port retractor 400 in closed and open positions, respectively. As can be seen in FIGS. 9 and 11, the port retractor 400 differs from the above described port retractor 20 as its leaves extend inwardly instead of substantially aligned with the body in the closed position. This provides for a reduced cross-sectional area at a distal tip, which can eliminate and/or reduce any required dilation of an incision before insertion of the port retractor 400. The port retractor 400 may be generally similar in other respects to the above described port retractor 20.

The port retractor 400 illustrated in FIGS. 9-12 includes a proximal portion 402 and a distal portion 404. The port retractor 400 includes a body 410, bending relief openings 406, malleable leaves 412, a bending opening 414, a distal tip 416, a mounting tab 418, and a mounting slot 420. In the illustrated embodiment, the body 410 is located proximate to the proximal end of the port retractor 400, and defines a substantially circular cross-section. In alternate embodiments, the body may define different shapes and/or include different numbers of leaves. As examples, a port retractor with inwardly tapered leaves in the closed position may define a polygonal perimeter (such as a square, rectangle, or octagon), or, as another example, an oval perimeter. The port retractor 400 includes four malleable leaves 412 extending from the body toward the distal end of the port retractor. Each malleable leaf 412 corresponds generally to a quadrant of the circle defined by the perimeter of the body 410. The malleable leaves 412 taper toward the distal end, or extend inwardly, maintaining a generally circularly shaped cross-sectional area that reduces in size distally. The port retractor 400 may also include a sheath (not shown) as discussed above. The mounting tab 418 and mounting slot 420 are configured to cooperate with a retractor frame (not shown) to facilitate mounting and securement of the port retractor 400 in place during a procedure.

The bending relief openings 406 improve the ease of bending the malleable leaves 412 by reducing the amount of material. Further, the shape of the gaps between the malleable leaves 412 may facilitate the inward taper in the closed position. For example, in the above discussed port retractor 20, the gaps running along the lengths of the leaves had substantially parallel edges when the leaves where substantially aligned with the body. In contrast, the gaps between the leaves 412 of the port retractor 400 may be triangular or wedge-shaped when the leaves 412 are substantially aligned with the body 410, thereby allowing the leaves 412 to be bent a desired distance inwardly without interfering with each other.

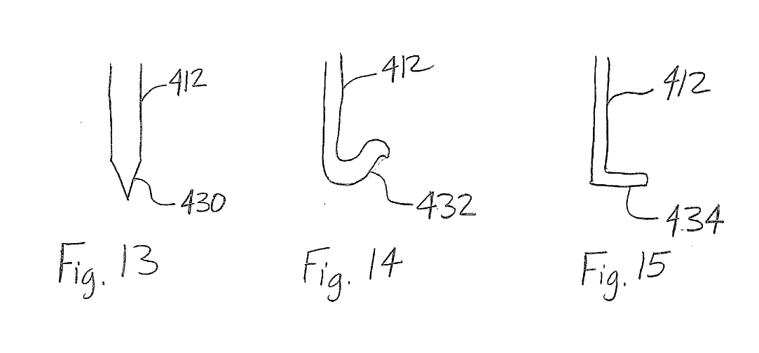

The distal tip 416 of the malleable leaf 412 of the port retractor 400 may include a feature designed to aid insertion of the port retractor, and/or aid retraction of tissue, and/or aid securement of the port retractor 400. For example, the distal tip 416 may include a tapered wedge 430, or chisel-like feature, as illustrated in FIG. 13. Such a tapered wedge 430 can be useful in streamlining the profile of the port retractor 400 to ease insertion into an incision, and/or improve engagement with a feature of a patient's anatomy to help secure and/or maintain the port retractor 400 in place during a procedure. The distal tip 416 may include a lip, such as a radiused bend 432 as shown in FIG. 14. Such a radiused bend 432 may bend outwardly and be sized and configured to help retract a portion of the patient's anatomy such as a blood vessel and help maintain the blood vessel in a position away from instruments being inserted through the port retractor 400 during a procedure. As another example, the distal tip 416 may include a generally perpendicular bend 434 as shown in FIG. 15. Further, the bend may be more or less than 90 degrees. Further still, the feature of the distal tip 416 may provide for mounting and/or securement to a portion of a patient's anatomy, such as a tab and an opening for using a bone screw or pin to mount the retractor's distal end to a portion of the patient's anatomy. Yet further still, such features of a distal tip 416 may be used with other configurations of port retractors, such as the above discussed port retractors 20 and 1000.

The port retractor 400 may be used generally similarly in many respects to the above described use of the port retractor 20. However, due to the inward taper of the malleable leaves 412 in the closed position, the port retractor 400 provides improved ease of insertion into an incision. For example, the port retractor 400 may be inserted into an incision without the use of dilation between the creation of the incision and the insertion of the port retractor 400.

While particular embodiments of the invention have been shown, it will be understood that the invention is not limited thereto since modifications may be made by those skilled in the art, particularly in light of the foregoing teaching.

What is claimed is:

1. A port retractor movable between a closed position and an open position for retracting tissue during a surgical procedure, the port retractor including
a body defining a perimeter; and
leaves extending distally from the body, the leaves being independently bendable;
wherein in the closed position, the leaves do not extend substantially outwardly from the body relative to the perimeter; and
wherein in the open position, at least one of the leaves extends outwardly relative to said perimeter from said body.

2. The port retractor of claim 1 further comprising a sheath attachable to and removable from at least a portion of the leaves, the sheath surrounding at least a portion of a distal portion of the leaves, the sheath being expandable with the leaves when the leaves are moved from the closed position to the open position and collapsible with the leaves when the leaves are moved from the open position to the closed position.

3. The port retractor of claim 1, wherein the body defines a substantially circular perimeter.

4. The port retractor of claim 1, wherein the body defines a substantially oval perimeter.

5. The port retractor of claim 1, wherein in the closed position, the leaves extend substantially aligned with the body.

6. The port retractor of claim 1, wherein in the closed position, the leaves extend inwardly from the body relative to the perimeter, wherein a cross-sectional area defined by the leaves at a distal end is smaller than a cross-sectional area defined by the leaves at a more proximal location.

7. The port retractor of claim 1, wherein the port refractor includes a plurality of opposed pairs of leaves.

8. The port refractor of claim 1, wherein at least one of the leaves includes a feature configured to cooperate with a feature of a bending tool for bending the at least one of the leaves.

9. A refractor system including
a port refractor movable between a closed position and an open position for refracting tissue during a surgical procedure, the port refractor including
a body defining a perimeter; and
leaves extending distally from the body, the leaves being independently bendable;
wherein in the closed position, the leaves do not extend substantially outwardly from the body relative to the perimeter; and
wherein in the open position, at least one of the leaves extends outwardly relative to said perimeter from said body; and
a bending tool configured for bending at least one of the leaves.

10. The port refractor system of claim 9 further comprising a sheath attachable to and removable from at least a portion of the leaves, the sheath surrounding at least a portion of a distal portion of the leaves, the sheath being expandable with the leaves when the leaves are moved from the closed position to the open position and collapsible with the leaves when the leaves are moved from the open position to the closed position

11. The port refractor system of claim 9 wherein at least one of the leaves and the bending tool include cooperating features configured to align and secure the at least one of the leaves and the bending tool during bending of the at least one of the leaves.

12. The port retractor system of claim 11 wherein the at least one of the malleable leaves includes a slot and the bending tool includes a hook configured to extend through the slot.

13. The port retractor system of claim 9 wherein the port retractor includes at least one pair of opposed leaves, and the bending tool includes two arms joined by a pivot, each of the leaves of the at least one pair of opposed leaves configured to be engaged by one of the two aims, wherein the bending tool is configured to engage each of the leaves of the at least one pair of opposed leaves simultaneously.

14. The port retractor system of claim 13 wherein each of the at least one pair of opposed leaves includes a slot and each of the two arms of the bending tool includes a hook configured to extend through one of the slots.

15. The port retractor system of claim 9, wherein in the closed position, the leaves extend inwardly from the body relative to the perimeter, wherein a cross-sectional area defined by the leaves at a distal end is smaller than a cross-sectional area defined by the leaves at a more proximal location.

16. A method of retracting tissue during a surgical procedure, the method including the steps of:
making an incision to provide access to a surgical site of interest;
inserting a port retractor in a closed position into the insertion, the port retractor including a body defining a perimeter and leaves extending distally from the body, the leaves being independently bendable, wherein in the closed position, the leaves do not extend substantially outwardly from the body relative to the perimeter, and wherein in an open position, at least one of the leaves extends outwardly relative to said perimeter from said body; and
bending at least one of the leaves to the open position using a bending tool that removably engages the at least one of the leaves.

17. The method of claim 16, further comprising the step of bending the at least one of the leaves back to the closed position for removal.

18. The method of claim 16, wherein in the closed position, the leaves extend inwardly from the body relative to the perimeter, wherein a cross-sectional area defined by the leaves at a distal end is smaller than a cross-sectional area defined by the leaves at a more proximal location, and wherein the inserting takes place after the step of making an incision without any intervening dilation.

19. The method of claim 16 wherein the port retractor includes a plurality of opposed pairs of leaves, and wherein leaves of at least one of the opposed pairs of malleable leaves are bent simultaneously.

20. The method of claim 16 wherein each of the leaves are bent independently.
